(19) United States
(12) Patent Application Publication
(10) Pub. No.: US 2011/0077436 A1
(43) Pub. Date: Mar. 31, 2011

(54) PRETREATMENT OF OILS AND/OR FATS

(75) Inventors: Jianhua YAO, Bartlesville, OK (US); Xiaochun XU, Bartlesville, OK (US); Dhananjay B. GHONASGI, Bartlesville, OK (US); Edward L. SUGHRUE, II, Bartlesville, OK (US); Yun BAO, Bartlesville, OK (US); Kathy A. SWALLOWS, Bartlesville, OK (US); Larry D. SWINNEY, Stillwater, OK (US); Scott A. SCHOLTEN, Katy, TX (US)

(73) Assignee: CONOCOPHILLIPS COMPANY, Houston, TX (US)

(21) Appl. No.: 12/877,328

(22) Filed: Sep. 8, 2010

Related U.S. Application Data
(60) Provisional application No. 61/246,704, filed on Sep. 29, 2009.
(60) Provisional application No. 61/246,732, filed on Sep. 29, 2009.

(51) Int. Cl. C07C 1/00

(52) U.S. Cl. 585/240

(57) ABSTRACT

Disclosed are methods for pretreating triglyceride containing material prior to contacting with a hydrotreating catalyst to produce fuel range hydrocarbons without causing reactor fouling or catalyst plugging.

### CROSS-REFERENCE TO RELATED APPLICATIONS

This application is a non-provisional application which claims benefit under 35 USC §119(e) to both U.S. Provisional Application Ser. No. 61/246,704 filed Sep. 29, 2009, entitled “PRETREATMENT OF OILS AND/OR FATS” and U.S. Provisional Application Ser. No. 61/246,732 filed Sep. 29, 2009, entitled “PRETREATMENT OF OILS AND/OR FATS FOR CONVERSION TO TRANSPORTATION FUEL” which are incorporated herein in their entirety.

### STATEMENT OF FEDERALLY SPONSORED RESEARCH

None

### FIELD OF THE INVENTION

The present invention relates generally to the pretreatment of triglycerides containing material prior to its conversion to fuel range hydrocarbons.

### BACKGROUND OF THE INVENTION

There is a national interest in the discovery of alternative sources of fuels and chemicals, other than from petroleum resources. As the public discussion concerning the availability of petroleum resources and the need for alternative sources continues, government mandates will require transportation fuels to include, at least in part, hydrocarbons derived from sources besides petroleum. As such, there is a need to develop alternative sources for hydrocarbons useful for producing fuels and chemicals.

One possible alternative source of hydrocarbons for producing fuels and chemicals is the natural carbon found in plants and animals, such as for example, oils and fats. These so-called “natural” carbon resources (or renewable hydrocarbons) are widely available, and remain a target alternative source for the production of hydrocarbons. For example, it is known that oils and fats, such as those contained in vegetable oil, can be processed and used as fuel. “Bio Diesel” is one such product and may be produced by subjecting a base vegetable oil to a transesterification process using methanol in order to convert the base oil to desired methyl esters. After processing, the products produced have very similar combustion properties as compared to petroleum-derived hydrocarbons. However, the use of Bio-Diesel as an alternative fuel has not yet been proven to be cost effective. In addition, Bio-Diesel often exhibits “gelling” thus making it unable to flow, which limits its use in pure form in cold climates.

Unmodified vegetable oils have also been used as additives in diesel fuel to improve the qualities of the diesel fuel, such as for example, the lubricity. However, problems such as injector coking and the degradation of combustion chamber conditions have been associated with these unmodified additives. Since cetane (C₁₆H₃₄), heptadecane (C₁₇H₃₆) and octadecane (C₁₈H₃₈) by definition have very good ignition properties (expressed as cetane rating), it is often desired to add paraffinic hydrocarbons in the C₁₆-C₁₈ range, provided that other properties of the additive, such as for example, viscosity, pour point, cloud point, etc., are congruent with those of the diesel fuel.

Processes for converting animal fat and vegetable oil into hydrocarbons have been achieved, such as, for example, contacting a diesel/vegetable oil mixture with a hydrotreating catalyst. However, oftentimes, animal fat and vegetable oils can contain significant amounts of solids, metals and phosphorus compounds and other impurities, which can cause catalyst deactivation and plugging of the reactor catalyst bed. As such, it is desirable to develop a process of pretreating the animal fat and vegetable oil prior to contacting them with a hydrotreating catalyst to produce a diesel boiling range hydrocarbons without causing reactor fouling or catalyst plugging.

### SUMMARY OF THE INVENTION

In one embodiment of the present invention, a process for treating a triglyceride containing feedstock comprising: a) providing a feedstock comprising at least one triglyceride; b) subjecting the feedstock to a heating zone to form treated feedstock A; c) subjecting the treated feedstock A to a separation device to form a treated feedstock B; and d) reacting the treated feedstock B with a hydrotreating catalyst in a reaction zone under a condition sufficient to produce a reaction product containing diesel boiling range hydrocarbons. The temperature in the heating zone is in the range of from about 40° C. to about 540° C., and the condition in the reaction zone includes a pressure in the range of from 100 to 2000 psig and a temperature in the range of from about 260° C. to about 430° C.

In another embodiment of the present invention, a process for treating a triglyceride containing feedstock comprising: a) providing a feedstock comprising at least one triglyceride; b) mixing the feedstock with a hydrocarbon boiling in the temperature range of from about 25° C. to about 760° C. to form a feedstock mixture; c) subjecting the feedstock mixture to a heating zone to form treated feedstock mixture A; d) subjecting the treated feedstock mixture A to a separation device to form a treated feedstock mixture B; and e) reacting the treated feedstock mixture B with a hydrotreating catalyst in a reaction zone under a condition sufficient to produce a reaction product containing diesel boiling range hydrocarbons. The temperature in the heating zone is in the range of from about 40° C. to about 540° C., and the condition in the reaction zone includes a pressure in the range of from 100 to 2000 psig and a temperature in the range of from about 260° C. to about 430° C. In yet another embodiment of the present invention, the step (c) is carried out in the presence of a co-feed gas.

In yet another embodiment of the present invention, a process for treating a triglyceride containing feedstock comprising: a) providing a feedstock comprising at least one triglyceride; b) maintaining the feedstock in a temperature sufficient to keep the feedstock in liquid form; c) settling the feedstock for a retention time to form a clear top layer and a bottom layer of sediment; and d) recovering and reacting the clear layer with a hydrotreating catalyst in a reaction zone under a condition sufficient to produce a reaction product containing diesel boiling range hydrocarbons. The temperature in step (b) is in the range of from about 30° C. to about 150° C., and the retention time in step (c) is at least 30 minutes, and the condition in the reaction zone includes a pressure in the range of from 100 to 2000 psig and a temperature in the range of from about 260° C. to about 430° C.

In yet another embodiment of the present invention, a process for treating a triglyceride containing feedstock and comprising: a) providing a feedstock comprising at least one triglyceride; b) subjecting the feedstock to a separation device to remove at least part of impurities from the feedstock and produce an effluent stream; c) reacting the effluent stream with a hydrotreating catalyst in a reaction zone under a condition sufficient to produce a reaction product containing diesel boiling range hydrocarbons. The condition in the reaction zone includes a pressure in the range of from 100 to 2000 psig and a temperature in the range of from about 260° C. to about 430° C.

### DETAILED DESCRIPTION OF THE INVENTION

In one embodiment of the present invention, a process for treating a triglyceride containing feedstock comprising: a) providing a feedstock comprising at least one triglyceride; b) subjecting the feedstock to a heating zone to form treated feedstock A; c) subjecting the treated feedstock A to a separation device to form a treated feedstock B; and d) reacting the treated feedstock B with a hydrotreating catalyst in a reaction zone under a condition sufficient to produce a reaction product containing diesel boiling range hydrocarbons. The temperature in the heating zone is in the range of from about 40° C. to about 540° C., and the condition in the reaction zone includes a pressure in the range of from 100 to 2000 psig and a temperature in the range of from about 260° C. to about 430° C.

In another embodiment of the present invention, a process for treating a triglyceride containing feedstock comprising: a) providing a feedstock comprising at least one triglyceride; b) mixing the feedstock with a hydrocarbon boiling in the temperature range of from about 25° C. to about 760° C. to form a feedstock mixture; c) subjecting the feedstock mixture to a heating zone to form treated feedstock mixture A; d) subjecting the treated feedstock mixture A to a separation device to form a treated feedstock mixture B; and e) reacting the treated feedstock mixture B with a hydrotreating catalyst in a reaction zone under a condition sufficient to produce a reaction product containing diesel boiling range hydrocarbons. The temperature in the heating zone is in the range of from about 40° C. to about 540° C., and the condition in the reaction zone includes a pressure in the range of from 100 to 2000 psig and a temperature in the range of from about 260° C. to about 430° C.

In yet another embodiment of the present invention, the step (c) is carried out in the presence of a co-feed gas. Generally, the co-feed gas is selected from the group consisting of hydrogen, nitrogen, helium, carbon monoxide, and carbon dioxide. In one embodiment, the co-feed gas can be hydrogen or nitrogen.

According to the embodiments above, a thermally treated feed can pass through a separation device before passing to the hydrotreating reaction zone, which will be described later in detail. Any suitable separation device capable of separating the solid from the triglyceride containing feed may be used. A separation device according to one embodiment of the current invention is a commercially available bag or cartridge filter with a pore size of at least 0.1 μm. In another embodiment with the feedstock being the inedible tallow, the first separation device of choice is a commercially available bag or cartridge filter with a pore size anywhere from 0.1 to 25 μm.

Generally, a treated feed after the above separation device can be contacted with a catalyst composition under a condition sufficient to produce a reaction product containing diesel boiling range hydrocarbons.

In yet another embodiment of the present invention, a process for treating a triglyceride containing feedstock comprising: a) providing a feedstock comprising at least one triglyceride; b) maintaining the feedstock in a temperature sufficient to keep the feedstock in liquid form; c) settling the feedstock for a retention time to form a clear top layer and a bottom layer of sediment; and d) recovering and reacting the clear layer with a hydrotreating catalyst in a reaction zone under a condition sufficient to produce a reaction product containing diesel boiling range hydrocarbons. The temperature in step (b) is in the range of from about 30° C. to about 150° C., and the retention time in step (c) is at least 30 minutes, and the condition in the reaction zone includes a pressure in the range of from 100 to 2000 psig and a temperature in the range of from about 260° C. to about 430° C. According to this embodiment of the invention, a feedstock comprising triglyceride was kept in liquid form by maintaining the feedstock in a temperature range from about 30 to 150° C. The feedstock is allowed to settle for at least 30 minutes to thereby form a clear top layer of treated feedstock and a bottom layer of sediment. The layer of the treated feedstock is recovered but not limited by separation funnel, decanting method, centrifugation, and etc. Further refereeing to this embodiment of the current invention, the bottom layer of sediment is selected from the group consisting of phosphorus, metals (e.g. alkali metals, alkaline earth metals), solids, proteins, bone materials, and any combinations thereof. The amounts of these compounds are generally in the range of from about 0 ppmw to about 10,000 ppmw. In addition, a treated feed, after the settling process can be contacted with a catalyst composition under a condition sufficient to produce a reaction product containing diesel boiling range hydrocarbons.

In yet another embodiment of the present invention, a process for treating a triglyceride containing feedstock and comprising: a) providing a feedstock comprising at least one triglyceride; b) subjecting the feedstock to a separation device to remove at least part of impurities from the feedstock and produce an effluent stream; c) reacting the effluent stream with a hydrotreating catalyst in a reaction zone under a condition sufficient to produce a reaction product containing diesel boiling range hydrocarbons. The condition in the reaction zone includes a pressure in the range of from 100 to 2000 psig and a temperature in the range of from about 260° C. to about 430° C. According to this embodiment of the invention, a feedstock comprising triglyceride is subject to a separation device wherein part of impurities are removed from the feedstock. Any suitable separation device capable of separating the solid from an oil phase feed may be used. The separation device according to one embodiment of the current invention is a commercially available bag or cartridge filter with a pore size of at least 0.1 μm. In another embodiment with the feedstock being the inedible tallow, the separation device of choice is a commercially available bag or cartridge filter with a pore size anywhere from 0.1 to 25 μm, which removes at least 50% of the impurities from the inedible tallow feedstock to produce the effluent stream. Further according to this embodiment of the invention, the impurities is selected from the group consisting of phosphorus, metals (e.g. alkali metals, alkaline earth metals), solids, proteins, bone materials, and any combinations thereof. The amounts of these compounds are generally in the range of from about 0 ppmw to about 10,000 ppmw. In addition, a treated feed, after the separation process can be contacted with a catalyst composition under a condition sufficient to produce a reaction product containing diesel boiling range hydrocarbons.

According to the invention in general, triglycerides or fatty acids of triglycerides, or mixtures thereof, may be converted to form a hydrocarbon mixture useful for liquid fuels and chemicals. The term, “triglyceride,” is used generally to refer to any naturally occurring ester of a fatty acid and/or glycerol having the general formula CH₂(OCOR₁)CH(OCOR₂)CH₂(OCOR₃), where R₁, R₂, and R₃ are the same or different, and may vary in chain length. Useful triglycerides in the present invention include, but are not limited to, triglycerides that may be converted to hydrocarbons when contacted under suitable reaction conditions. Examples of triglycerides useful in the present invention include, but are not limited to, animal fats (e.g. poultry grease, edible or inedible beef fat also referred as tallow, milk fat, and the like), vegetable oils (e.g. soybean, corn oil, peanut oil, sunflower seed oil, coconut oil, babassu oil, grape seed oil, poppy seed oil, almond oil, hazelnut oil, walnut oil, olive oil, avocado oil, sesame, oil, tall oil, cottonseed oil, palm oil, ricebran oil, canola oil, cocoa butter, shea butter, butyrospermum, wheat germ oil, illipe butter, meadowfoam, seed oil, rapeseed oil, borage seed oil, linseed oil, castor oil, vernoia oil, tung oil, jojoba oil, ongokea oil, algae oil, jatropha oil, yellow grease such as those derived from used cooking oils, and the like), and mixtures and combinations thereof.

Generally, the triglyceride may be present in an amount in the range of from about 0.1 to about 100 percent, based on the total weight percent of the feed. The triglyceride can also be present in an amount in the range of from about 50 weight percent to about 99.9 weight percent based on the total weight of the mixture. The triglyceride can also be present in the feed in an amount of 100 weight percent.

Generally, the triglyceride contains amounts of metal compounds and phosphorus compounds. The elements that the triglyceride contains are generally selected from the group consisting of phosphorus, alkali metals, alkaline earth metals and combinations thereof. The amounts of these compounds are generally in the range of from about 0 ppmw to about 10,000 ppmw.

In according to the invention, triglyceride starting materials may be processed alone or in combination with other hydrocarbons. The hydrocarbons generally boil at a temperature of from about 25° C. to about 760° C. Examples of suitable hydrocarbons include middle distillate fuels. Middle distillate fuels generally contain hydrocarbons that boil in the middle distillate boiling range in the range from about 150° C. to about 400° C. Typical middle distillates may include for example, jet fuel, kerosene, diesel fuel, light cycle oil, atmospheric gas oil, and vacuum gas oil. If a middle distillate feed is employed in the process of the present invention, the feed generally may contain a mixture of hydrocarbons having a boiling range (ASTM D86) of from about 150° C. to about 400° C. In addition, the middle distillate feed may have a mid-boiling point (ASTM D86) of greater than about 175° C. A middle distillate feed employed in one embodiment of the present invention is diesel fuel. In addition, one or more triglycerides can mix with a middle distillate feed. In addition to middle distillate fuels, other suitable hydrocarbons include, but are not limited to, gasoline, naphtha, and atmospheric tower bottom.

In one embodiment of the present invention the temperature in the heating zone is in the range of from about 40° C. to about 540° C. In another embodiment of the present invention, the temperature in the heating zone is in the range of from about 120° C. to about 430° C., and in yet another embodiment of the present invention, the temperature in the heating zone is in the range of from about 200° C. to about 400° C.

Useful catalyst compositions in the present invention include catalysts effective in the conversion of triglycerides to hydrocarbons when contacted under suitable reaction conditions. Examples of suitable catalysts include hydrotreating catalysts. The term “hydrotreating” as used herein, generally describes a catalyst that is capable of utilizing hydrogen to accomplish saturation of unsaturated materials, such as aromatic compounds. Examples of hydrotreating catalysts useful in the present invention include, but are not limited to, materials containing compounds selected from Group VI and Group VIII metals, and their oxides and sulfides. Examples of hydrotreating catalysts include but are not limited to alumina supported cobalt-molybdenum, nickel sulfide, nickel-tungsten, cobalt-tungsten and nickel-molybdenum.

The metal of the catalyst useful in the present invention is usually distributed over the surface of a support in a manner than maximizes the surface area of the metal. Examples of suitable support materials for the hydrogenation catalysts include, but are not limited to, silica, silica-alumina, aluminum oxide (Al₂O₃), silica-magnesia, silica-titania and acidic zeolites of natural or synthetic origin. The metal catalyst may be prepared by any method known in the art, including combining the metal with the support using conventional means including but not limited to impregnation, ion exchange and vapor deposition. In an embodiment of the present invention, the catalyst contains molybdenum and cobalt supported on alumina or molybdenum and nickel supported on alumina.

This process in accordance with the present invention can be carried out in any suitable reaction zone that enables intimate contact of the treated feed and control of the operating conditions under a set of reaction conditions that include total pressure, temperature, liquid hourly space velocity, and hydrogen flow rate. The catalyst can be added first to the reactants and thereafter, fed with hydrogen. In the present invention, either fixed bed reactors or fluidized bed reactors can be used. As used herein, the term “fluidized bed reactor” denotes a reactor wherein a fluid feed can be contacted with solid particles in a manner such that the solid particles are at least partly suspended within the reaction zone by the flow of the fluid feed through the reaction zone and the solid particles are substantially free to move about within the reaction zone as driven by the flow of the fluid feed through the reaction zone. As used herein, the term “fluid” denotes gas, liquid, vapor and combinations thereof.

Generally, the reaction conditions at which the reaction zone is maintained generally include a temperature in the range of from about 260° C. to about 430° C. Preferably, the temperature is in the range of from about 310° C. to about 370° C.

In accordance with the present invention, regardless of whether a fixed or fluidized bed reactor is used, the pressure is generally in the range of from about 100 pounds per square inch gauge (psig) to about 2000 psig. Generally, in a fixed bed reactor, the pressure is in the range of from about 100 psig to about 1500 psig. In a fixed bed reactor, the pressure can also be about 600 psig. In a fluidized bed reactor, the pressure is generally in the range of from about 400 psig to about 750 psig, and can also be about 500 psig.

The following examples are presented to further illustrate the present invention and are not to be construed as unduly limiting the scope of this invention.

### Example 1

A mixture of soybean oil and diesel was fed into a heated tube operated at a temperature of about 330° C. and a pressure of 700 psig (there was no co-feed gas present). The mixture was then passed through a filter and sent to a hydrotreating reactor containing a hydrotreating catalyst. Table 2 below shows that the hydrotreating reactor experienced no pressure drop, unlike when the same mixture is fed through a hydrotreating reactor without the pre-treatment.

TABLE 1

Reactor Configuration

| | Hydrotreating | Heated Tube/Filter/ |
|---|---|---|
| | Reactor Only | Hydrotreating Reactor |
| Time On-Stream, | 50 | 100 |
| hrs | | |
| Reactor Pressure | 100 | none |
| Drop, psig | | |

### Example 2

A mixture of beef tallow and diesel was fed into a heated tube operated at a temperature of about 300° C. The resulting liquid was passed through a filter and then sent to a hydrotreating reactor containing a hydrotreating catalyst. Table 3 below shows that the hydrotreating reactor experienced no pressure drop after 20 days on stream while the untreated feed led to 90 psig pressure drop after 1 day on stream operation.

TABLE 2

Reactor Configuration

| | Hydrotreating | Heated Tube/Filter/ |
|---|---|---|
| | Reactor Only | Hydrotreating Reactor |
| Time On-Stream, | 1 | 20 |
| days | | |
| Reactor Pressure | 90 | None |
| Drop, psig | | |

### Example 3

A tallow sample was kept at 65° C. (in liquid form) for one week. A layer of brownish sediment was settled at the bottom of a glass container. The top clear tallow liquid was decanted out and sent to a hydrotreating reactor containing a hydrotreating catalyst. Table 1 below shows that the hydrotreating reactor experienced no pressure drop after 10 days on stream while the untreated feed led to 90 psig pressure drop after 1 day on stream operation. Therefore, using the decanted clear tallow sample as feed can reduce reactor fouling issue.

TABLE 3

| | Reactor Configuration | |
|---|---|---|
| | 20% untreated technical | 20% decanted clear |
| | tallow with 80% diesel/ | tallow with 80% diesel/ |
| | Hydrotreating Reactor | Hydrotreating Reactor |
| Time On-Stream, | 1 | 10 |
| days | | |
| Reactor Pressure | 90 | none |
| Drop, psig | | |

### Example 4

A tallow sample was passed through a 2 μm filter. The filtered tallow was thereafter sent to a hydrotreating reactor containing a hydrotreating catalyst. Table 2 below shows that the hydrotreating reactor experienced no pressure drop after 14 days on stream while the untreated feed led to 90 psig pressure drop after 1 day on stream operation.

TABLE 4

| | Reactor Configuration | |
|---|---|---|
| | 20% untreated technical | 20% filtered tallow |
| | tallow with 80% diesel/ | (2 μm) with 80% diesel/ |
| | Hydrotreating Reactor | Hydrotreating Reactor |
| Time On-Stream, | 1 | 14 |
| days | | |
| Reactor Pressure | 90 | none |
| Drop, psig | | |

While this invention has been described in detail for the purpose of illustration, it should not be construed as limited thereby but intended to cover all changes and modifications within the spirit and scope thereof.

That which is claimed:

1. A process for treating a triglyceride containing feedstock comprising:
(a) providing a feedstock comprising at least one triglyceride;
(b) subjecting said feedstock to a heating zone to form treated feedstock A;
(c) subjecting said treated feedstock A to a separation device to form a treated feedstock B; and
(d) reacting said treated feedstock B with a hydrotreating catalyst in a reaction zone under a condition sufficient to produce a reaction product containing diesel boiling range hydrocarbons,
wherein said temperature in said heating zone is in the range of from about 40° C. to about 540° C., and
wherein said condition in said reaction zone includes a pressure in the range of from 100 to 2000 psig and a temperature in the range of from about 260° C. to about 430° C.

2. The process of claim 1, wherein said triglyceride is present in said feedstock in an amount in the range of from about 0.01 to about 100 weight percent based on the total weight of said feedstock.

3. The process of claim 1, wherein said triglyceride is selected from the group consisting of vegetable oil, soybean oil, yellow grease, animal fats and mixtures thereof.

4. The process of claim 1, wherein said feedstock further comprises elements selected from the group consisting of phosphorus, alkali metals, alkaline earth metals, and combinations thereof.

5. The process of claim 1, wherein said hydrotreating catalyst comprises nickel and molybdenum.

6. The process of claim 1, wherein said hydrotreating catalyst comprises cobalt and molybdenum.

7. The process of claim 1, wherein said separation device is a filter with a pore size of at least 0.1 μm.

8. A process for treating a triglyceride containing feedstock comprising:
(a) providing a feedstock comprising at least one triglyceride;
(b) mixing said feedstock with a hydrocarbon boiling in the temperature range of from about 25° C. to about 760° C. to form a feedstock mixture;
(c) subjecting said feedstock mixture to a heating zone to form treated feedstock mixture A;
(d) subjecting said treated feedstock mixture A to a separation device to form a treated feedstock mixture B; and
(e) reacting said treated feedstock mixture B with a hydrotreating catalyst in a reaction zone under a condition sufficient to produce a reaction product containing diesel boiling range hydrocarbons,
wherein said temperature in said heating zone is in the range of from about 40° C. to about 540° C., and
wherein said condition in said reaction zone includes a pressure in the range of from 100 to 2000 psig and a temperature in the range of from about 260° C. to about 430° C.

9. The process of claim 8, where in said step (c) is carried out in the presence of a co-feed gas.

10. The process of claim 8, wherein said triglyceride is present in said feedstock in an amount in the range of from about 0.01 to about 100 weight percent based on the total weight of said feedstock.

11. The process of claim 8, wherein said triglyceride is selected from the group consisting of vegetable oil, soybean oil, yellow grease, animal fats and mixtures thereof.

12. The process of claim 8 wherein said feedstock further comprises elements selected from the group consisting of phosphorus, alkali metals, alkaline earth metals, and combinations thereof.

13. The process of claim 8, wherein said hydrotreating catalyst comprises nickel and molybdenum.

14. The process of claim 8, wherein said hydrotreating catalyst comprises cobalt and molybdenum.

15. The process of claim 8, wherein said separation device is a filter with a pore size of at least 0.1 μm.

16. The process of claim 8, wherein said hydrocarbon boiling in the temperature range of from about 25° C. to about 760° C. is selected from the group consisting of gasoline, naphtha, jet fuel, kerosene, diesel fuel, light cycle oil, vacuum gas oil, atmospheric gas oil, atmospheric tower bottom, and combinations of any two or more thereof.

17. The process in accordance with claim 9 wherein said co-feed gas is selected from the group consisting of hydrogen, nitrogen, helium, carbon monoxide, and carbon dioxide.

18. A process for treating a triglyceride containing feedstock comprising:
(a) providing a feedstock comprising at least one triglyceride;
(b) maintaining said feedstock in a temperature sufficient to keep said feedstock in liquid form;
(c) settling said feedstock for a retention time to form a clear top layer and a bottom layer of sediment; and
(d) recovering and reacting said clear layer with a hydrotreating catalyst in a reaction zone under a condition sufficient to produce a reaction product containing diesel boiling range hydrocarbons,
wherein said temperature in step (b) is in the range of from about 30° C. to about 150° C.,
wherein said retention time in step (c) is at least 30 minutes, and
wherein said condition in said reaction zone includes a pressure in the range of from 100 to 2000 psig and a temperature in the range of from about 260° C. to about 430° C.

19. The process of claim 18, wherein said triglyceride is present in said feedstock in an amount in the range of from about 0.01 to about 100 weight percent based on the total weight of said feedstock.

20. The process of claim 18, wherein said triglyceride is selected from the group consisting of vegetable oil, soybean oil, yellow grease, animal fats and mixtures thereof.

21. The process of claim 18 wherein said feedstock further comprises elements selected from the group consisting of phosphorus, alkali metals, alkaline earth metals, and combinations thereof.

22. The process of claim 18, wherein said hydrotreating catalyst comprises nickel and molybdenum.

23. The process of claim 18, wherein said hydrotreating catalyst comprises cobalt and molybdenum.

24. The process of claim 18, wherein said bottom layer of sediment is selected from the group consisting of phosphorus, metals, solids, proteins, bone materials, and any combinations thereof.

25. The process of claim 18, wherein said clear top layer maybe separated from said bottom layer of sediment by a separation method selected from a group consisting of separation funnel, decanting method, or centrifugation method.

26. A process for treating a triglyceride containing feedstock and comprising:
(a) providing a feedstock comprising at least one triglyceride;
(b) subjecting said feedstock to a separation device to remove at least part of impurities from said feedstock and produce an effluent stream;
(c) reacting said effluent stream with a hydrotreating catalyst in a reaction zone under a condition sufficient to produce a reaction product containing diesel boiling range hydrocarbons,
wherein said condition in said reaction zone includes a pressure in the range of from 100 to 2000 psig and a temperature in the range of from about 260° C. to about 430° C.

27. The process of claim 26, wherein said triglyceride is present in said feedstock in an amount in the range of from about 0.01 to about 100 weight percent based on the total weight of said feedstock.

28. The process of claim 26, wherein said triglyceride is selected from the group consisting of vegetable oil, soybean oil, yellow grease, animal fats and mixtures thereof.

29. The process of claim 26 wherein said feedstock further comprises elements selected from the group consisting of phosphorus, alkali metals, alkaline earth metals, and combinations thereof.

30. The process of claim 26, wherein said hydrotreating catalyst comprises nickel and molybdenum.

31. The process of claim 26, wherein said hydrotreating catalyst comprises cobalt and molybdenum.

32. The process of claim 26, wherein said separation device is a filter with a pore size of at least 0.1 μm.

33. The process of claim 26, wherein said impurities is selected from the group consisting of phosphorus, metals, solids, proteins, bone materials, and any combinations thereof.
